(19) United States
(12) Patent Application Publication
(10) Pub. No.: US 2023/0402136 A1
(43) Pub. Date: Dec. 14, 2023

(54) TRANSFORMER-BASED GRAPH NEURAL NETWORK TRAINED WITH STRUCTURAL INFORMATION ENCODING

(71) Applicant: Microsoft Technology Licensing, LLC, Redmond, WA (US)

(72) Inventors: Shuxin ZHENG, Beijing (CN); Yu SHI, Beijing (CN); Tie-Yan LIU, Beijing (CN)

(73) Assignee: Microsoft Technology Licensing, LLC, Redmond, WA (US)

(21) Appl. No.: 17/806,075

(22) Filed: Jun. 8, 2022

(51) Int. Cl. G16C 20/70; G16C 60/00; G16C 20/50; G06N 3/04; G06N 5/04

(52) U.S. Cl. CPC G16C 20/70; G16C 60/00; G16C 20/50; G06N 3/04; G06N 5/04

(57) ABSTRACT

A computing system is provided, including a processor configured to, during a training phase, provide a training data set, including a pre-transformation molecular graph and post-transformation energy parameter value representing an energy change in a molecular system following an energy transformation. The pre-transformation graph includes a plurality of normal nodes connected by edges representing a distance and a bond between a pair of the normal nodes. The processor is further configured to encode structural information in each pre-transformation molecular graph as learnable embeddings, the structural information describing the relative positions of the atoms represented by the normal nodes. The structural information includes an edge encoding representing a type of bond between a pair of normal nodes in each pre-transformation molecular graph, and a spatial encoding representing a shortest path distance along the edges between a pair of normal nodes in each pre-transformation molecular graph.

### BACKGROUND

In the field of computational chemistry, computer-based techniques have been developed to predict molecular properties through computer simulations. These molecular properties can have a wide-ranging impact on the appearance and function of a molecule or material, and thus are of keen interest in a wide variety of fields. For example, in the field of drug design, changes in molecular properties can affect the efficacy of a drug. In the field of drug discovery, molecular properties can affect the potential for a material found in nature to be used for therapeutic purposes. In the field of quantum chemistry, quantum-mechanical calculation of electronic contributions to physical and chemical properties of molecules and materials is a fundamental area of inquiry. As discussed below, opportunities remain for improvements in computational methods for predicting molecular properties, which would have application beyond the field of computational chemistry.

### SUMMARY

To address the issues discussed herein, computerized systems and methods are provided. In one aspect, the computerized system includes a processor configured to, during a training phase, provide a training data set including a plurality of training data pairs, each of the training data pairs including a pre-transformation molecular graph and post-transformation energy parameter value representing an energy change in a molecular system following an energy transformation, in which the pre-transformation molecular graph includes a plurality of normal nodes connected by edges, each normal node representing an atom in the molecular system. The processor is further configured to encode structural information in each pre-transformation molecular graph as learnable embeddings, the structural information describing the relative positions of the atoms represented by the normal nodes. The structural information includes an edge encoding representing a type of bond between a pair of the normal nodes in each pre-transformation molecular graph, and a spatial encoding representing a shortest path distance along the edges between the pair of the normal nodes in each pre-transformation molecular graph. The processor is further configured to input training data set to a transformer-based graph neural network to thereby train the transformer-based graph neural network to perform an inference at inference time.

These techniques are not limited to molecular graphs, but may be applied to other types of graphs that contain structural information. For example, these techniques may be applied to a social graph that models a social network, a map that models a network of locations, or a knowledge graph that models knowledge sources connected by references, as some examples.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows a schematic view of a computing system including a transformer-based graph neural network, during a training phase in which a training data set is used to train the transformer-based graph neural network to perform an inference at inference time, according to one example implementation of the present disclosure.

FIG. 2 shows a schematic view of an example of the training data set of FIG. 1, including a pre-transformation molecular graph and post-transformation energy parameter value, in which the pre-transformation molecular graph includes a plurality of normal nodes connected by edges and each normal node represents an atom in the molecular system.

FIG. 3 shows a schematic view of an example internal configuration of a transformer including an encoder and feed forward network, of the transformer-based graph neural network of the system of FIG. 1.

FIG. 4 shows a schematic view of structural information in the form of centrality encoding, spatial encoding, and edge encoding, being fed into the transformer-based graph neural network of the system of FIG. 1.

FIG. 5 shows a schematic view of a computing system including a trained transformer-based graph neural network configured to, during an inference phase, predict an inference-time post-transformation energy parameter value based on an inference-time pre-transformation molecular graph input via the trained transformer-based graph neural network of the computing system of FIG. 1.

FIGS. 6-9 are tables comparing the performance of the system of FIG. 1 with graph neural networks of other configurations on four different predictive tasks relating to four different datasets.

FIG. 10 is a table showing the results of an ablation study performed on different configurations of the system of FIG. 1.

FIG. 11 shows a flowchart of a computerized method according to one example implementation of the present disclosure.

FIG. 12 shows an example computing environment according to which the embodiments of the present disclosure may be implemented.

### DETAILED DESCRIPTION

Computer-based techniques have been developed to predict molecular properties through computer simulations. For example, Density Functional Theory (DFT) is a powerful and widely used quantum physics calculation technique that can in many cases accurately predict various molecular properties such as the shape of molecules, reactivity, responses by electromagnetic fields, etc. However, DFT is time-consuming and computationally intensive, often taking up to several hours even for a single model of a simple molecule on a conventional processor. For many complex systems, computing exact DFT solutions is not practical on current hardware. This currently presents a barrier to predicting molecular properties.

Design Principles

In view of the issues discussed above, a computing system utilizing a transformer-based graph neural network is provided. The computing system has applicability to predicting molecular properties of molecular systems, as well as to predicting other parameters of other types of systems that can be represented as graphs. The following discussion provides an overview of the theoretical underpinnings and design principles upon which the transformer-based graph neural network has been conceived. This discussion is followed by a detailed description of specific example embodiments of a transformer-based graph neural network.

The transformer-based graph neural network according to the present disclosure is trained using deep learning techniques to receive a graph as input and output a predicted scalar value. The graph may take the form G=(V,E), which denotes a graph G having nodes V and edges E, where V={v₁, v₂, . . . , vₙ}, n=|V| is the number of nodes. A feature vector may be provided for each node. For example, the feature vector of node vᵢ is denoted xᵢ. Feature vectors encode features of each node.

The transformer-based graph neural network may follow a learning schema that iteratively updates the representation of a node in a pre-transformation molecular graph by aggregating representations of its first or higher-order neighbors. Herein, hᵢ^{(l)} is the representation of vᵢ at the l-th layer and hᵢ⁽⁰⁾=xᵢ. The l-th iteration of aggregation could be characterized by an AGGREGATE-COMBINE step as follows:

aᵢ^{(l)}=AGGREGATE^{(l)}({hⱼ^{(l-1)}:j∈ [symbol] (vᵢ)}),hᵢ^{(l)}=COMBINE^{(l)}(hᵢ^{(l-1)},aᵢ^{(l)}) (1)

wherein [symbol] (vᵢ) is the set of first or higher-order neighbors of vᵢ. The AGGREGATE function is used to gather the information from neighbors. Suitable aggregation functions include MEAN, MAX, SUM. The goal of the COMBINE function is to fuse the information from neighbors into the node representation. In addition, for graph representation tasks, a READOUT function is designed to aggregate node features hᵢ^{(L)} of the final iteration into the representation h_{G} of the entire graph G:

h_{G}=READOUT({hᵢ^{(L)})|vᵢ∈G}) (2)

READOUT can be implemented by a simple permutation invariant function such as summation or a graph-level pooling function, for example.

The transformer architecture of the transformer-based graph neural network of the present disclosure may include one or more transformer layers. Each transformer layer has two parts: a self-attention module and a position-wise feed-forward network (FFN). H=[h₁^{T}, . . . , hₙ^{T}]^{T}∈R^{n×d} denotes the input of self-attention module where d is the hidden dimension and hᵢ∈R^{1×d} is the hidden representation at position i. The input H is projected by three matrices W_{Q} ∈R^{d×d}^{_{K}}, W_{κ}∈R^{d×d}^{_{κ}} and W_{V} ∈R^{d×d}^{_{V}} to the corresponding representations Q, K, V. The self-attention is calculated as:

$$\\ \begin{matrix}Q = {HW}_{Q} , K = {HW}_{K} , V = {HW}_{V} & ( 3 )\end{matrix}$$

$$\\ \begin{matrix}A = \frac{{QK}^{T}}{\sqrt{{d}_{K}}} , Attn ( H ) = softmax ( A ) V & ( 4 )\end{matrix}$$

where A is a matrix capturing the similarity between queries and keys. For simplicity, a single-head self-attention is described, and it is assumed that dK=dV=d. However, in practice a multi-head attention layer may be used. Bias terms are omitted for simplicity of explanation.

In Eq. 4, the attention distribution is calculated based on the semantic correlation between nodes. However, node centrality, which can measure how important a node is in the graph, can be a strong signal for graph understanding. Such information is neglected in conventional attention calculations for graph neural networks. In the transformer-based graph neural network of the present disclosure, centrality may be calculated in terms of the degree of each node. In one specific example, a centrality encoding is utilized that assigns to each node two real-valued embedding vectors according to the indegree and outdegree of the node. As the centrality encoding is applied to each node, it is added to the vector of node features, as follows.

hᵢ⁽⁰⁾=xᵢ+z_{deg}_{^{−}}₍ᵥ_{ᵢ}₎⁻+z_{deg}_{⁺}₍ᵥ_{ᵢ}₎⁺ (5)

where z⁻,z⁺∈R^{d} are learnable embedding vectors specified by the indegree deg⁻(vᵢ) and outdegree deg⁺(vᵢ) respectively. For undirected graphs, deg⁻(vᵢ) and outdegree deg⁺(vᵢ) could be unified to deg(vᵢ). By using centrality encoding in the input, the softmax attention can catch the node importance signal in the queries and the keys. Therefore, the trained model can capture both the semantic correlation and the node importance, based on its centrality, in the attention mechanism.

An advantage of the transformer architecture is its global receptive field. In each transformer layer, each token can attend to the information at any position and then process its representation. But this operation has a problematic byproduct that the model has to explicitly specify different positions or encode the positional dependency (such as locality) in the layers. For sequential data, such as sentences of words, the transformer input can be labeled with sequence position using an embedding (i.e., absolute positional encoding) or the transformer input can be encoded with the relative distance of any two positions (i.e., relative positional encoding).

However, for graphs, nodes are not arranged as a sequence. They can lie in a multi-dimensional spatial space and are linked by edges. To encode the structural information of a graph in the transformer-based graph neural network of the present disclosure, spatial encoding is utilized. Concretely, for any graph G, a function Ø(vᵢ, vⱼ): V×V→R measures the spatial relation between vᵢ and vⱼ in graph G. The function Ø can be defined by the connectivity between the nodes in the graph. Herein, Ø(vᵢ, vⱼ) represents the distance between vᵢ and vⱼ if the two nodes are connected. Typically, the distance is expressed as the shortest path distance (SPD), which may be expressed in terms of the number of edges on the shortest path, or may be weighted according to edge weights for each edge along the path. If not, the output of Ø is set to be a predetermined value, i.e., −1. Each (feasible) output value is assigned a learnable scalar which will serve as a bias term in the self-attention module. Denoting Aᵢⱼ as the (i,j)-element of the Query-Key product matrix A, the following expression may be obtained:

$$\\ \begin{matrix}{A}_{ij} = \frac{( {h}_{i} {W}_{Q} ) {( {h}_{i} {W}_{K} )}^{T}}{\sqrt{d}} + {b}_{∅ ( {v}_{i} , {v}_{j} )} & ( 6 )\end{matrix}$$

where b_{Ø(vi,vj)} is a learnable scalar indexed by Ø(vᵢ, vⱼ), and shared across all layers.

There are several technical benefits of the proposed transformer-based graph neural network described herein. First, compared to conventional graph neural networks, where the receptive field is restricted to neighbors, as shown in Eq. (6), the transformer layer provides global information such that each node can attend to all other nodes in the graph. Second, by using b_{Ø(vi,vj)}, each node in a single transformer layer can adaptively attend to all other nodes according to the graph structural information. For example, if b_{Ø(vi,vj)} is learned to be a decreasing function with respect to Ø(vᵢ, vⱼ), for each node, the model will likely pay more attention to the nodes near it and pay less attention to the nodes far away from it.

In many graph tasks, edges also have structural features, e.g., in a molecular graph, atom pairs may have features describing the type of bond between them. To capture this structural information, edge encoding may be used. There are two conventional edge encoding methods, each with its attendant technical drawbacks. In the first method, the edge features are added to the associated nodes' features. In the second method, for each node, its associated edges' features will be used together with the node features in the aggregation. However, such ways of using edge feature only propagate the edge information to its associated nodes, and thus the attention that can be given to those features is limited. As a result, the whole graph may fail to learn sufficiently from such edge information.

To better encode edge features into the attention layers, transformer-based graph neural network of the present disclosure may utilize the following edge encoding method. The attention mechanism estimates correlations for each node pair (vᵢ, vⱼ), and the edges connecting them should be considered in the correlation. For each ordered node pair (vᵢ, vⱼ), a shortest path SPᵢⱼ=(e₁, e₂, . . . , e_{N}) from vᵢ to vⱼ is determined, and an average of the dot-products of the edge feature and a learnable embedding along the path is calculated. This method of edge encoding incorporates edge features via a bias term to the attention module. Concretely, the (i,j)-element of A in Eq. (3) is modified further with the edge encoding cᵢⱼ as:

$$\\ \begin{matrix}{A}_{ij} = \frac{( {h}_{i} {W}_{Q} ) {( {h}_{i} {W}_{K} )}^{T}}{\sqrt{d}} + {b}_{∅ ( {v}_{i} , {v}_{j} )} + {c}_{ij} , where & ( 7 )\end{matrix}$$

$$\\ {c}_{ij} = \frac{1}{N} {\sum }_{n = 1}^{N} {{x}_{{e}_{n}} ( {ω}_{n}^{E} )}^{T}$$

where xₑ_{ₙ} is the feature of the n-th edge eₙ in SPᵢⱼ, ωₙ^{E} ∈R^{d}^{_{E}} is the n-th weight embedding, and d_{E} is the dimensionality of edge feature.

Layer normalization (LN) may be applied before the multi-head self-attention (MHA) and the feed-forward blocks (FFN) instead of after. This modification leads to more effective optimization. In particular, for the FFN sub-layer, the dimensionality of input, output, and the inner-layer(s) are set to the same dimension d. We formally characterize the transformer layer as follows:

h′^{(l)}=MHA(LN(h^{(l-1)}))+h^{(l-1)} (8)

h^{(l)}=FFN(LN(h′^{(l)}))+h′^{(l)} (9)

A predetermined node referred to as a virtual node [VNode] is added to the graph, and the virtual node is connected to each other normal node in the graph individually (i.e., is fully connected by unique edges). In the AGGREGATE-COMBINE step, the representation of [VNode] has been updated as normal nodes in graph, and the representation of the entire graph hG would be the node feature of the virtual node in the final layer. Since the virtual node is connected to all other nodes in graph, the distance of the shortest path is 1 (assuming no weighting) for any Ø([VNode], vⱼ) and Ø(vᵢ, [VNode]), although the connection is not physical. To distinguish the connection of physical and virtual edges, all spatial encodings for b_{Ø([VNode], v}_{ⱼ}₎ and b_{Ø(v}_{ᵢ}_{, [VNode])} are reset to a distinct learnable scalar.

### Example Embodiments

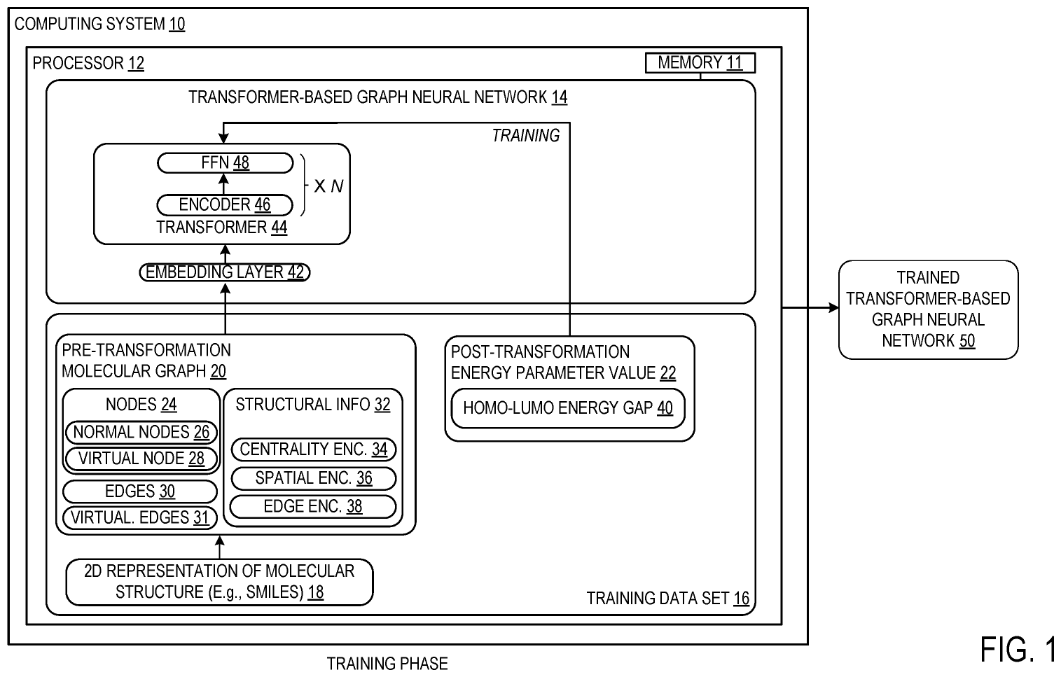

In accordance with principles discussed above, a specific example embodiment of a transformer-based graph neural network according to the present disclosure will now be described, with reference to FIGS. 1-12. FIG. 1 shows a schematic view of a computing system 10 including a transformer-based graph neural network 14, during a training phase in which a training data set 16 is used to train the transformer-based graph neural network 14 to perform an inference at inference time, according to one example implementation of the present disclosure. The computing system 10 may include one or more processors 12 configured to execute instructions using associated memory 11 to perform the functions and processes of the computing system 10 described herein. For example, the computing system 10 may include a cloud server platform including a plurality of server devices, and the one or more processors 12 may be one processor of a single server device, or multiple processors of multiple server devices. The computer system 10 may also include one or more client devices in communication with the server devices, and one or more of processors 12 may be situated in such a client device. Below, the functions of computing system 10 as executed by processor 12 are described by way of example, and this description shall be understood to include execution on one or more processors distributed among one or more of the devices discussed above.

Computing system 10 is configured to, during a training phase, train the transformer-based graph neural network 14 to perform an inference at inference time. Initially, the computing system 10 is configured to obtain or produce a 2D representation of molecular structure 18 in a format such as the SMILES (Simplified Molecular Input Line Entry System) format. Based on the 2D representation of molecular structure 18, the processor 12 of the computing system 10 is configured to provide, e.g., by computationally generating or reading from a stored location in memory, a training data set 16 including a plurality of training data pairs. Each of the training data pairs includes a pre-transformation molecular graph 20 and post-transformation energy parameter value 22 representing an energy change in a molecular system following an energy transformation which may be due to molecular relaxation of the molecular system. In one specific example, the transformation energy parameter value may be a value indicating a HOMO-LUMO energy gap. Other energy parameter values are also contemplated, as are applications to graph systems other than molecular systems, as described below.

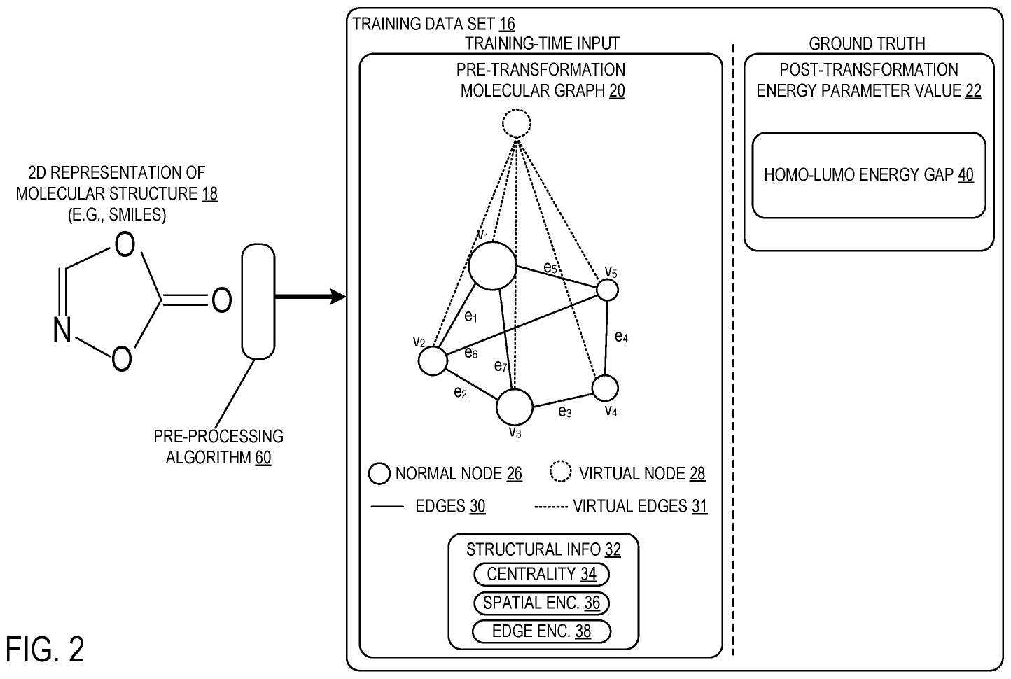

Turning briefly to FIG. 2, the training data set 16 is further explained. FIG. 2 shows a schematic view of an example of the training data set 16 of FIG. 1, including the pre-transformation molecular graph 20 and post-transformation energy parameter value 22. As shown, the pre-transformation molecular graph 20 includes a plurality of normal nodes 26 connected by edges 30. Each normal node 26 represents an atom in the molecular system. As discussed briefly above, the pre-transformation molecular graph 20 is created based on a 2D representation of molecular structure 18, such as SMILES, via a pre-processing algorithm 60. Each pre-transformation molecular graph 20 further includes one virtual node 28 fully connected by virtual edges 31 to all normal nodes 26 of the respective pre-transformation molecular graph 20. It will be appreciated that the difference between the virtual node 28 and normal nodes 26 is that the normal nodes represent atoms whereas the virtual node is provided for computation purposes only, and does not represent any physical component of the molecular system. Other detail regarding the design principles of the virtual node is discussed above. In the depicted example, the pre-transformation molecular graph 20 includes five normal nodes representing atoms (v₁, v₂, v₃, v₄, v₅) connected by edges 30 (e₁, e₂, e₃, e₄, and e₅). The pre-transformation molecular graph 20 further includes one virtual node 28 fully connected via the virtual edges 31 to each normal node (v₁, v₂, v₃, v₄, v₅). In the pre-transformation molecular graph 20, the normal nodes are not necessarily fully connected to other normal nodes, but the virtual node is fully connected to all normal nodes.

Turning back to FIG. 1, the processor 12 is further configured to encode structural information 32, which describes the relative positions of the atoms represented by the normal nodes 26, in each pre-transformation molecular graph 20 as learnable embeddings. The encoded structural information 32 is represented as a learnable scalar bias term in a self-attention layer of an encoder 46 of a transformer 44 of the transformer-based graph neural network 14, as discussed below. In the depicted example, the encoded structural information 32 includes a centrality encoding 34, a spatial encoding 36, and an edge encoding 38, as introduced generally above. In one example implementation, the centrality encoding 34 is embedded in (i.e., is provided as an embedding for) each normal node 26 of each pre-transformation molecular graph 20 and represents a degree (i.e., number of edge connections) of each normal node 26. In one specific implementation, the pre-transformation molecular graph 20 may be a directed graph, and the degree may be represented as an indegree and outdegree of each normal node. Thus, the centrality encoding 34 assigned to each normal node 26 may be represented as two real-valued embedding vectors that contain values for an indegree and an outdegree of the respective normal node 26. The edge encoding 38 represents a type of bond between each pair of normal nodes 26 in each pre-transformation molecular graph 20. For example, the edge encoding may indicate an ionic, covalent, or metallic bond. The spatial encoding 36 may represent a shortest path distance along the edges between each pair of normal nodes 26 in each pre-transformation molecular graph 20. In one example, the shortest path distance is the smallest number of edges connecting two nodes. In another example, each edge has a weight associated with it, representing relative distance, energy, or other parameter of the edge, etc., and the shortest path distance is a weighted shortest path distance computed as the sum of the weights of the shortest path of edges between two nodes in the graph.

The processor 12 is further configured to input the training data set 16 to a transformer-based graph neural network 14 to train the transformer-based graph neural network 14 to perform an inference at inference time. Within the training data set 16, there are a plurality of training data pairs, each pair including an instance of the pre-transformation molecular graph 20 and an associated instance of the post transformation energy parameter value 22. The pre-transformation molecular graph 20 is put through an embedding layer 42, which produces an embedding representation (i.e., embeddings) of the graph. The embeddings are produced by a program that is configured to convert atomic information in the 2D representation of the molecular structure to a numerical value representing the atomic information. The embedding representation of the pre-transformation molecular graph 20 is fed into an encoder 46 of a transformer 44 of the transformer-based graph neural network 14 to generate an encoded representation in the form of an attention vector. The attention vector generated by the encoder 46 is transmitted to a feed-forward network 48 which includes one or more fully connected hidden layers that perform deep learning based on ground truth output that is received during training. Specifically, the post-transformation energy parameter value 22, which may be a HOMO-LUMO energy gap 40, is supplied to the transformer 44 of the transformer-based graph neural network 14 as a ground truth output to train the transformer-based graph neural network 14 in order to output a predicted inference-time post-transformation energy parameter value at an inference time. Following the training phase, the processor of the computing system 10 is further configured to output a trained transformer-based graph neural network 50, which is used at an inference time on the computing system 10 or another suitable computing system.

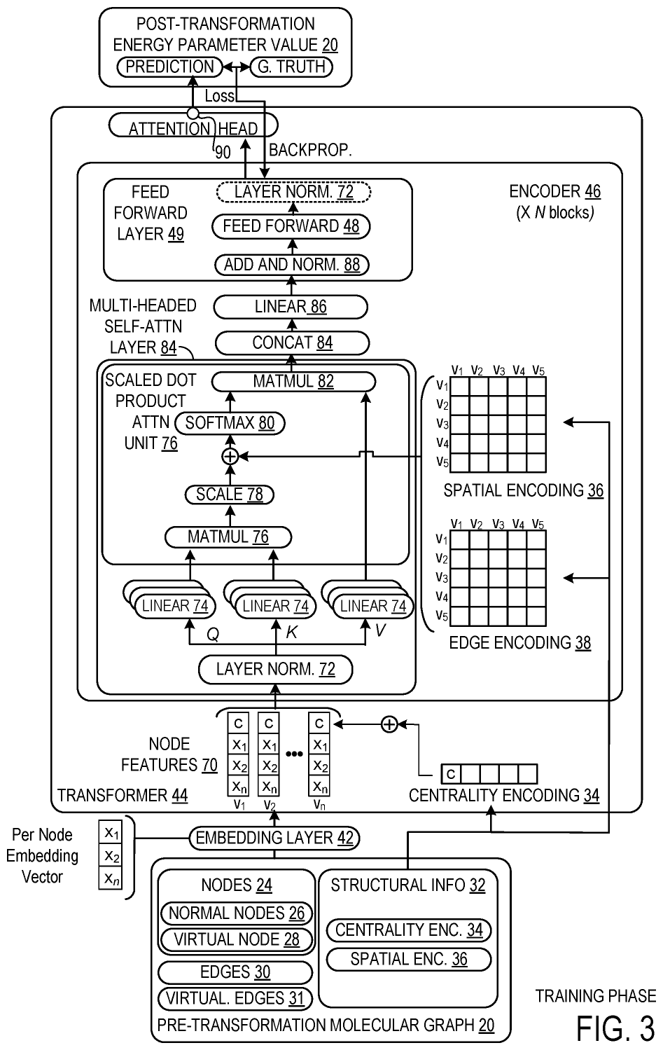

FIG. 3 shows a schematic view of an example internal configuration of the transformer 44 including the encoder 46 of the transformer-based graph neural network 14 of the system of FIG. 1. As shown in FIG. 3, nodes 24 of the pre-transformation molecular graph are passed through the embeddings layer 42, which generates a vector of embeddings for each node. The structural information 32 is already expressed in a parameterized form and thus is not converted to embeddings by the embeddings layer. Rather the structural information 32 is passed to the encoder 46 in its encoded numeric form. It will be appreciated that within structural information 32, centrality encodings 34 are node-wise structural information, that is, are computed on a per-node basis. For this reason, the centrality encoding 34 for each node is concatenated to the embedding vector for that respective node, thereby creating a concatenated vector of node features 70. The edge encodings 38, which represent the bond between nodes, and spatial encodings 36, which represent distance between nodes, are not node-wise information, and for this reason are inputted into the scalar dot product unit 76 within the multi-headed self-attention layer 84. The node features 70 are passed through a normalization layer 72 before passing through a linear projection layer 74 in which vectors for queries Q, keys K, and values V are projected into the matrix multiplication layer 76, which performs dot product multiplication on the keys and query values. The output is then scaled by scaling layer 78, and appended with spatial encodings 36 and edge encodings 38 before being passed through softmax layer 80. Finally, the linear projection of the values vector is multiplied by dot product multiplication in the matrix multiplication layer 82, to produce the output of the scaled dot product attention unit 76. This process occurs in parallel for each attention head of the multiple attention heads, and the results of all attention heads are concatenated in concatenation layer 84 and their linear projection is transmitted to the addition and normalization layer 88 of feed forward layer 49, and then again to the feed forward neural network 48. The output of the feed forward neural network 48 is routed through a regressor node 90 of an attention head, the regressor node 90 being configured to output a scalar value. It will be appreciated that during training, a prediction of the scalar value is compared to ground truth for the scalar value, and loss function is used to train the feed forward network using a suitable backpropagation algorithm. The multi-headed self-attention layer 84 and feed forward layer 49 form one block of encoder 46, and it will be appreciated that multiple blocks of encoder 46 may be chained together.

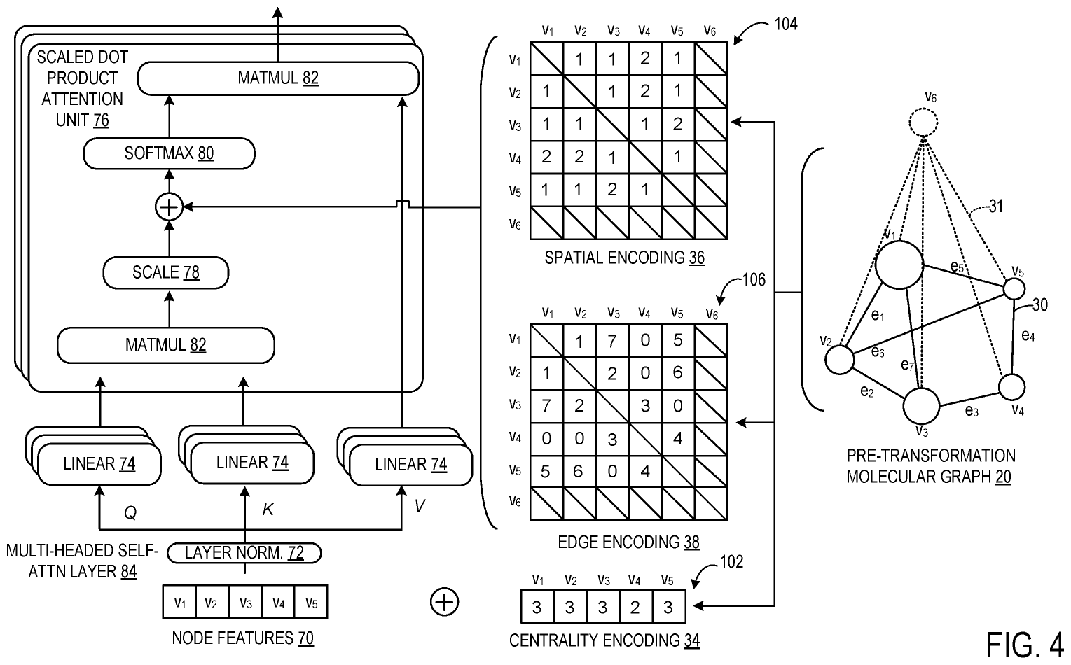

FIG. 4 illustrates a detailed schematic view with example values for the centrality encodings 34, spatial encodings 36, and edge encodings 38 that are fed into the multi-headed self-attention layer 84 of the encoder 46 of FIG. 3. In the depicted example, the pre-transformation molecular graph 20 includes five normal nodes 26 (v₁, v₂, v₃, v₄, v₅) connected by edges 30 (e₁, e₂, e₃, e₄, and e₅) and one virtual node (v₆) fully connected to each normal node 26. As shown in example centrality encoding vector 102, the centrality encoding is computed as 3, 3, 3, 2, and 3 for v₁, v₂, v₃, v₄, and v₅ respectively, since the normal node v₁ is connected to three other normal nodes (v₂, v₃, v₅), the normal node v₂ is connected to three other normal nodes (v₁, v₃, v₅), the normal node v₃ is connected to three other normal nodes (v₁, v₂, v₄), the normal node v₄ is connected to two other normal nodes (v₃, v₅), and the normal node v₅ is connected to three other normal nodes (v₁, v₂, v₄). As shown in in example spatial encoding vector 104, the spatial encoding 36, which may represent the shortest path distance along the edges between each pair of normal nodes 26, is computed for v₁, v₂, v₃, v₄, and v₅. For example, the spatial encoding 36 for v₁-v₂ is computed as 1 since v₁ and v₂ are directly connected by one edge, while the spatial encoding 36 for v₁-v₄ is computed as 2 since v₁ and v₄ are indirectly connected through v₃ by two edges. Finally, as shown in edge encoding vector 106, the edge encoding 38, which may represent a type of bond between each pair of normal nodes 26, is computed for v₁, v₂, v₃, v₄, and v₅. For instance, the edge encoding 38 for v₁-v₂ is 1 as v₁ and v₂ are connected by the edge e₁, and the edge encoding 38 for v₁-v₃ is 7 as v₁ and v₃ are connected by the edge e₇, and the edge encoding 38 for v₁-v₄ is 0 as v₁ and v₄ are not connected by any edge. The spatial encoding 36 and edge coding 38 for v₆ is not computed since v₆ is the virtual node 28. As discussed above, the structural information 32 is fed into the multi-headed self-attention layer 84 of the encoder 46, with node-wise centrality encodings 34 being concatenated to the vector of node features 70 and the spatial encodings 36 and edge encodings 38 being concatenated to the scaled product of the query and key attention vectors, prior to softmax layer 80 in the scaled dot product attention unit 76 of the multi-headed attention layer 84. Doing so increases the parameter space of the attention vector, enabling the model to attend to the structural features as well as the node-specific features such as atom type, etc. in the pre-transformation molecular graph 20 during deep learning. This increases the expressiveness of the model.

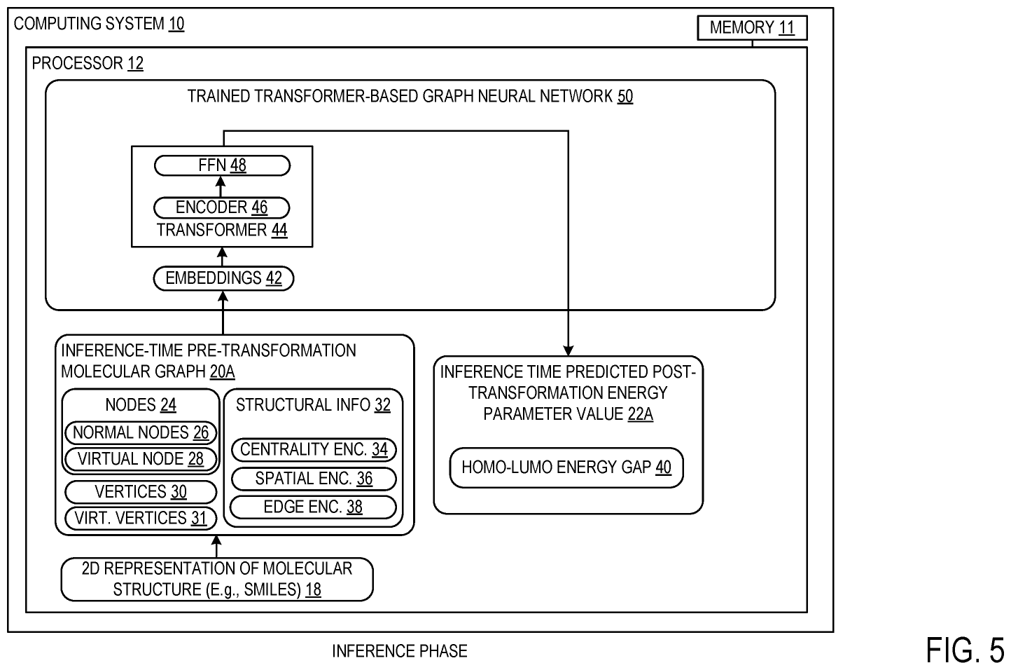

FIG. 5 shows a schematic view of a computing system 10 including a trained transformer-based graph neural network 50 that has been trained by the methods heretofore discussed, to be configured to, during an inference phase, predict an inference-time post-transformation energy parameter value 22A based on an inference-time pre-transformation molecular graph 20A input via the trained transformer-based graph neural network 50 of the computing system 10 of FIG. 1. To perform the inference at inference time, the processor is configured to receive inference-time input of an inference-time pre-transformation molecular graph 20A at the transformer-based graph neural network 50, process the inference-time input, and output the inference-time post-transformation energy parameter value 22A, which may be the HOMO-LUMO energy gap 40 as discussed above, based on the inference-time pre-transformation molecular graph 20A. The structural information 32 including the centrality encoding 32, spatial encoding 36, and edge encoding 38, is encoded in the inference-time pre-transformation molecular graph 20A. The inference-time pre-transformation molecular graph 20A including the structural information 32 is first put through an embeddings layer 42 to convert the nodes into embeddings, which in turn are concatenated with the node-wise centrality encoding 34 as discussed above, prior to input into transformer 44. The concatenated vector of node features (including the embeddings and centrality encodings) is fed into an encoder 46 of the transformer 44 of the trained transformer-based graph neural network 50, which also receives the spatial encoding 36 and edge encoding 38. In turn, the trained transformer-based graph neural network 50 outputs a predicted inference-time post-transformation energy parameter value 22A representing, for example, a HOMO-LUMO energy gap 40.

Technical advantages of the configuration of the transformer-based graph neural network 14 discussed herein will now be explained. First, the architecture described herein has been shown to offer superior expressiveness as compared to conventional GNN models that merely use AGGREGATE and COMBINE steps, by choosing proper weights and distance function p. The reason for this is that the spatial encoding described herein enables the self-attention function to distinguish the neighbor set N(vᵢ) of node vᵢ so that the softmax function can calculate mean statistics over N(vⱼ). Further, by knowing the degree of a node due to centrality encoding, the mean over neighbors can be translated to the sum over neighbors. With the multiple heads in the self-attention layer and the feed forward network, representations of vᵢ and N(vᵢ) can be processed separately and combined together downstream. Further by using the spatial encoding described herein (e.g., shortest path distance), the transformer-based graph neural network described herein can exceed the results of conventional message passing GNNs whose expressive power is no more than the 1-Weisfeiler-Lehman (WL) test, enabling systems built according to the present disclosure to distinguish graphs that the 1-WL test cannot.

In addition to the improved expressiveness as compared to conventional GNNs, the use of self-attention and the virtual node can significantly improve the performance of existing GNNs. Conceptually, the benefit of the virtual node is that it can aggregate the information of the whole graph and then propagate it to each node. However, a naive addition of a fully connected virtual node to a graph can potentially lead to inadvertent over-smoothing of information propagation. The approach described herein instead demonstrates that such a graph-level aggregation and propagation operation can be naturally fulfilled by a self-attention layer as described herein without additional encodings. Due to the self-attention that each node can attend to all other nodes, the graph can simulate a graph-level READOUT operation to aggregate information from the entire graph. Further, the disclosed configurations do not encounter the problem of over-smoothing, which makes the improvement scalable. A predetermined node for graph readout may be provisioned to take advantage of this.

Experimental Results

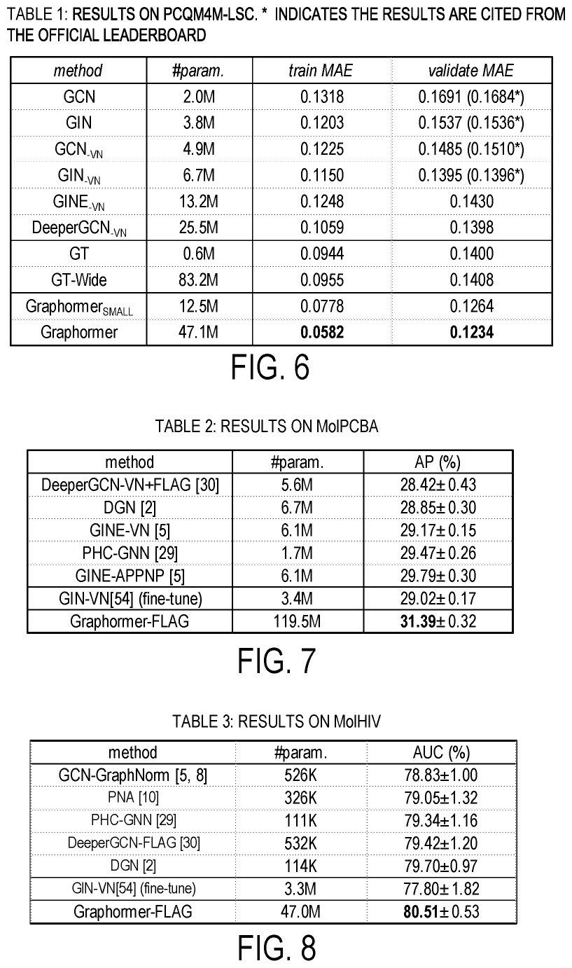

FIG. 6 illustrates experimental results of the system of FIG. 1 compared with other GNNs involved in the OGB-LSC quantum chemistry regression (i.e., PCQM4M-LSC) challenge, which was a prediction challenge that used a large graph-level prediction dataset that contained more than 3.8M graphs in total. The system of FIG. 1 is referred to as GRAPHORMER in FIGS. 6-9. GRAPHORMER is compared to Graph Convolutional Networks (GCN) and Graph Isomorphism Network (GIN), and their variants with virtual nodes (-VN). These GNNs achieved the state-of-the-art valid and test Mean Absolute Error (MAE) on the official leaderboard for the Open Graph Benchmark-Large Scale Challenge (OGB-LSG) challenge. In addition, the results compare GIN's multi-hop variant, and the 12-layer deep graph network DeeperGCN. The results listed in FIG. 6 further compare GRAPHORMER with the recent transformer-based graph model Graph Transformer (GT).

The results in FIG. 6 include two model sizes: GRAPHORMER (L=12, d=768), and GRAPHORMER_{SMALL} (L=6, d=512). Both the number of attention heads in the multi-head attention layer and the dimensionality of edge features d_{E} are set to 32. AdamW was used as the optimizer, and hyper-parameter ∈ was set to 1e-8 and (β1, β2) to (0.99,0.999). The peak learning rate was set to 2e-4 (3e-4 for Graphormer_{SMALL}) with a 60 k-step warm-up stage followed by a linear decay learning rate scheduler. The total training steps were 1M. The batch size was set to 1024. All models were trained on 8 NVIDIA V100 GPUS for about 2 days.

Table 1 of FIG. 6 summarizes performance comparisons on the PCQM4M-LSC dataset. From the table, GIN_{-VN} achieves the previous state-of-the-art validate MAE of 0.1395. The original implementation of GT employs a hidden dimension of 64 to reduce the total number of parameters. For comparison, the result of enlarging the hidden dimension to 768, denoted by GT_{Wide}, is also included, which leads to a total number of parameters of 83.2M. Although both GT and GT-Wide do not outperform GIN_{-VN} and DeeperGCN_{-VN}, a performance gain was not observed along with the growth of parameters of GT.

Compared to the previous conventional GNN architectures, GRAPHORMER noticeably surpasses GIN_{-VN} by a large margin, e.g., 11.5% relative validate MAE decline. By using the ensemble with ExpC [55], a 0.1200 MAE on complete test set was achieved by GRAPHORMER, ranking first in the graph-level track in OGB Large-Scale Challenge. As mentioned above, GRAPHORMER does not encounter the problem of over-smoothing, i.e., the train and validate error keeps going down along with the growth of depth and width of the models.

FIGS. 7-9 illustrate results for three other predictive tasks: ogbg-molhiv, ogbg-molpcba, and ZINC. The performance of GRAPHORMER was investigated on commonly used graph-level prediction tasks, namely, OGB (OGBG-MolPCBA, OGBG-MolHIV), and benchmarking-GNN (ZINC). The transferable capability of a GRAPHORMER model pre-trained on OGB-LSC (i.e., PCQM4M-LSC) was investigated. The model configurations, hyper-parameters, and the pre-training performance of pre-trained GRAPHORMER used for MolPCBA and MolHIV are different from the models used in the previous subsection. For benchmarking-GNN, we train an additional GraphormerSLIM (L=12, d=80, total param.=489K) from scratch on ZINC.

Considering that the pre-trained GRAPHORMER leverages external data, for a comparison on OGB datasets the relative performance for fine-tuning GIN_{-VN} pre-trained on the PCQM4M-LSC dataset is reported, which achieves the previous state-of-the-art valid and test MAE on that dataset. Tables 2, 3 and 4 in FIGS. 7-9 summarize performance of GRAPHORMER as compared to other GNNs on MolHIV, MolPCBA and ZINC datasets. In particular, GT and SAN in Table 4 are recently proposed Transformer-based GNN models. GRAPHORMER consistently and significantly outperforms previous conventional GNNs on all three datasets by a large margin. Other pre-trained GNNs do not achieve competitive performance.

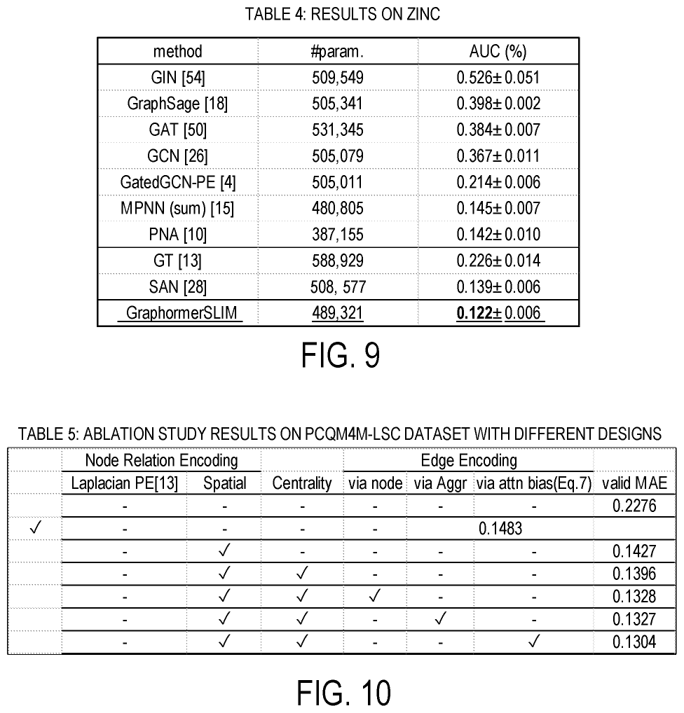

FIG. 10 illustrates the result of ablation studies performed on the system of FIG. 1. A series of ablation studies were performed on the effect of different configurations of GRAPHORMER, on PCQM4M-LSC dataset. The ablation results are included in Table 5 of FIG. 10. To save the computation resources, the Transformer models in table 5 have 12 layers, and are trained for 100K iterations.

Regarding node relation encoding, previously used positional encodings (PE) are compared to the spatial encoding of the present disclosure. There are various PEs employed by previous Transformer-based GNNs, e.g., Weisfeiler-Lehman-PE (WL-PE) and Laplacian PE. The transformer architecture with the spatial encodings described herein outperformed the counterparts built on the positional encoding, which demonstrates the effectiveness of using spatial encoding to capture the node spatial information.

Regarding centrality encoding, the edge encoding of the present disclosure (denoted as via attn bias) is compared to two commonly used edge encodings used to incorporate edge features into GNNs, denoted as via node and via Aggr in Table 5. As shown in the table, the gap of performance is minor between the two conventional methods, but the edge encoding disclosed herein performs significantly better, which indicates that edge encoding as attention bias is more effective for transformers to capture spatial information on edges.

As discussed above, the systems and methods described herein have applicability outside of the field of computational chemistry, on graphs in general, which encode structural information about the data they represent in their structure. In such a case, the processor described above may be configured to, more generally, during a training phase, provide a training data set including a plurality of training data pairs, each of the training data pairs including a pre-transformation graph and post-transformation parameter value representing a change in a system modeled by the pre-transformation graph following a transformation. The pre-transformation graph may include a plurality of normal nodes connected by edges, each normal node representing a location in the system. The processor may be configured to encode structural information in each pre-transformation graph as learnable embeddings, the structural information describing the relative positions of the locations represented by the normal nodes. The structural information may include an edge encoding representing a type of connection between each pair of normal nodes in each pre-transformation graph, and a spatial encoding representing a shortest path distance along the edges between each pair of normal nodes in each pre-transformation graph. The processor may further be configured to input the training data set to a transformer-based graph neural network to thereby train the transformer-based graph neural network to perform an inference at inference time. In one particular example, the pre-transformation graph may be a social graph that models a social network of friends. In such an example, the post-transformation parameter value may be an affinity ranking between two users of the social network. In another example, the pre-transformation graph may be a map that models a network of locations connected by roads or railways or other travelways. In this example, the post-transformation parameter value may be a ranking value of a route between two locations on the map. In another example, the pre-transformation graph may be a knowledge graph that models knowledge sources connected by references, and the post-transformation parameter value may be an influence score indicating relative influence of a knowledge source on the graph.

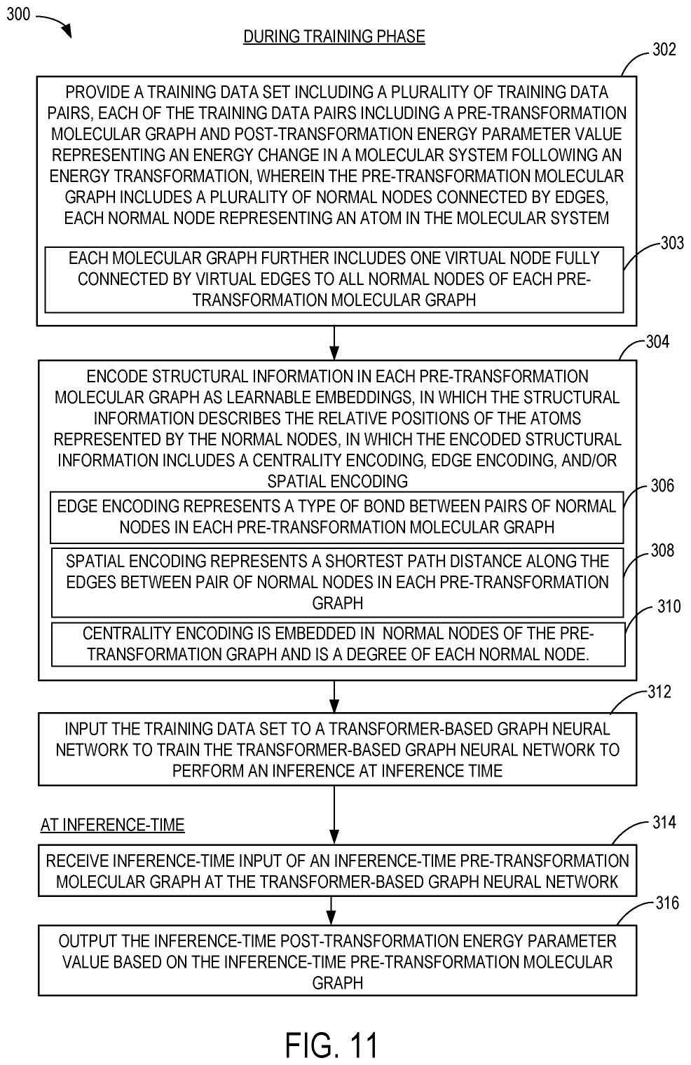

FIG. 11 shows a flowchart of a computerized method 300 according to one example implementation of the present disclosure. Method 300 may be implemented by the hardware and software of computing system 10 described above, or by other suitable hardware and software. At step 302, the method 300 may include, during a training phase, providing a training data set including a plurality of training data pairs, each of the training data pairs including a pre-transformation molecular graph and post-transformation energy parameter value representing an energy change in a molecular system following an energy transformation, wherein the pre-transformation graph includes a plurality of normal nodes connected by edges, each normal node representing an atom in the molecular system. As indicated at 303, each molecular graph further includes one virtual node fully connected by virtual edges to all normal nodes of the respective pre-transformation molecular graph.

At step 304, the method may further include encoding structural information in each pre-transformation molecular graph as learnable embeddings, in which the structural information describes the relative positions of the atoms represented by the normal nodes. As shown at 306 and 308, the structural information may include an edge encoding representing a type of bond between at least one of a plurality of bonded pairs of the normal nodes in each pre-transformation molecular graph, and a spatial encoding representing a shortest path distance along the edges between at least one of the plurality of pairs of normal nodes in each pre-transformation molecular graph. The pairs of normal nodes that include the edge encoded are bonded to each other, whereas the pairs of normal nodes that include the spatial encoding may or may not be bonded to each other. Typically, the structural information includes an edge encoding representing a type of bond between each of the plurality of pairs of the normal nodes in each pre-transformation molecular graph, and a spatial encoding representing a shortest path distance along the edges between each of the plurality of pairs of normal nodes in each pre-transformation molecular graph. Further as indicated at 310, the structural information may include a centrality encoding embedded for at least one (and typically for each) normal node of each pre-transformation molecular graph. The centrality encoding may be expressed as a degree of at least one (and typically of each) normal node. Where the graph is a directed graph, the degree may include an indegree and an outdegree.

At step 312, the method may further include inputting training data set to a transformer-based graph neural network to train the transformer-based graph neural network to infer a post-transformation molecular graph based on an inference-time input of a pre-transformation molecular graph.

At step 314, the method may further include, to perform the inference at inference-time, receiving inference-time input of an inference-time pre-transformation molecular graph at the transformer-based graph neural network. At step 316, the method may further include outputting the inference-time post-transformation energy parameter value based on the inference-time pre-transformation molecular graph.

In some embodiments, the methods and processes described herein may be tied to a computing system of one or more computing devices. In particular, such methods and processes may be implemented as a computer-application program or service, an application-programming interface (API), a library, and/or other computer-program product.

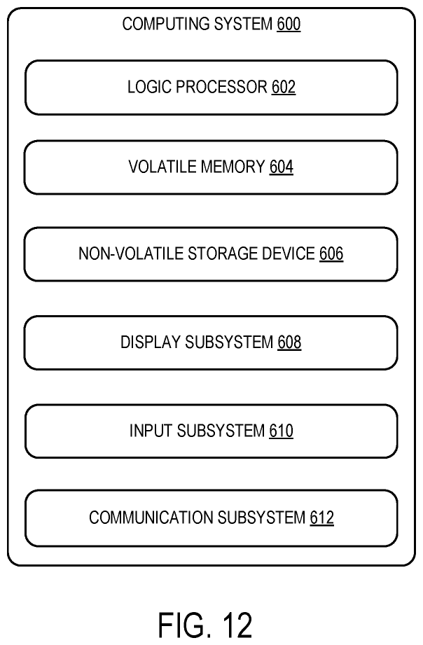

FIG. 12 schematically shows a non-limiting embodiment of a computing system 600 that can enact one or more of the methods and processes described above. Computing system 600 is shown in simplified form. Computing system 600 may embody the computer system 10 described above and illustrated in FIG. 1. Computing system 600 may take the form of one or more personal computers, server computers, tablet computers, home-entertainment computers, network computing devices, gaming devices, mobile computing devices, mobile communication devices (e.g., smart phone), and/or other computing devices, and wearable computing devices such as smart wristwatches and head mounted augmented reality devices.

Computing system 600 includes a logic processor 602 volatile memory 604, and a non-volatile storage device 606. Computing system 600 may optionally include a display subsystem 608, input subsystem 610, communication subsystem 612, and/or other components not shown in FIG. 12.

Logic processor 602 includes one or more physical devices configured to execute instructions. For example, the logic processor may be configured to execute instructions that are part of one or more applications, programs, routines, libraries, objects, components, data structures, or other logical constructs. Such instructions may be implemented to perform a task, implement a data type, transform the state of one or more components, achieve a technical effect, or otherwise arrive at a desired result.

The logic processor may include one or more physical processors (hardware) configured to execute software instructions. Additionally or alternatively, the logic processor may include one or more hardware logic circuits or firmware devices configured to execute hardware-implemented logic or firmware instructions. Processors of the logic processor 602 may be single-core or multi-core, and the instructions executed thereon may be configured for sequential, parallel, and/or distributed processing. Individual components of the logic processor optionally may be distributed among two or more separate devices, which may be remotely located and/or configured for coordinated processing. Aspects of the logic processor may be virtualized and executed by remotely accessible, networked computing devices configured in a cloud-computing configuration. In such a case, these virtualized aspects are run on different physical logic processors of various different machines, it will be understood.

Non-volatile storage device 606 includes one or more physical devices configured to hold instructions executable by the logic processors to implement the methods and processes described herein. When such methods and processes are implemented, the state of non-volatile storage device 606 may be transformed—e.g., to hold different data.

Non-volatile storage device 606 may include physical devices that are removable and/or built-in. Non-volatile storage device 606 may include optical memory (e.g., CD, DVD, HD-DVD, Blu-Ray Disc, etc.), semiconductor memory (e.g., ROM, EPROM, EEPROM, FLASH memory, etc.), and/or magnetic memory (e.g., hard-disk drive, floppy-disk drive, tape drive, MRAM, etc.), or other mass storage device technology. Non-volatile storage device 606 may include nonvolatile, dynamic, static, read/write, read-only, sequential-access, location-addressable, file-addressable, and/or content-addressable devices. It will be appreciated that non-volatile storage device 606 is configured to hold instructions even when power is cut to the non-volatile storage device 606.

Volatile memory 604 may include physical devices that include random access memory. Volatile memory 604 is typically utilized by logic processor 602 to temporarily store information during processing of software instructions. It will be appreciated that volatile memory 604 typically does not continue to store instructions when power is cut to the volatile memory 604.

Aspects of logic processor 602, volatile memory 604, and non-volatile storage device 606 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The terms “module,” “program,” and “engine” may be used to describe an aspect of computing system 600 typically implemented in software by a processor to perform a particular function using portions of volatile memory, which function involves transformative processing that specially configures the processor to perform the function. Thus, a module, program, or engine may be instantiated via logic processor 602 executing instructions held by non-volatile storage device 606, using portions of volatile memory 604. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

When included, display subsystem 608 may be used to present a visual representation of data held by non-volatile storage device 606. The visual representation may take the form of a graphical user interface (GUI). As the herein described methods and processes change the data held by the non-volatile storage device, and thus transform the state of the non-volatile storage device, the state of display subsystem 608 may likewise be transformed to visually represent changes in the underlying data. Display subsystem 608 may include one or more display devices utilizing virtually any type of technology. Such display devices may be combined with logic processor 602, volatile memory 604, and/or non-volatile storage device 606 in a shared enclosure, or such display devices may be peripheral display devices.

When included, input subsystem 610 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity; and/or any other suitable sensor.

When included, communication subsystem 612 may be configured to communicatively couple various computing devices described herein with each other, and with other devices. Communication subsystem 612 may include wired and/or wireless communication devices compatible with one or more different communication protocols. As non-limiting examples, the communication subsystem may be configured for communication via a wireless telephone network, or a wired or wireless local- or wide-area network, such as a HDMI over Wi-Fi connection. In some embodiments, the communication subsystem may allow computing system 600 to send and/or receive messages to and/or from other devices via a network such as the Internet.

The following paragraphs discuss several aspects of the present disclosure. According to one aspect of the present disclosure, a computing system is provided. The system may include a processor configured to, during a training phase, provide a training data set including a plurality of training data pairs, each of the training data pairs including a pre-transformation molecular graph and post-transformation energy parameter value representing an energy change in a molecular system following an energy transformation, in which the pre-transformation molecular graph includes a plurality of normal nodes connected by edges, each normal node representing an atom in the molecular system. The processor may be further configured to encode structural information in each pre-transformation molecular graph as learnable embeddings, in which the structural information describes the relative positions of the atoms represented by the normal nodes. The structural information may include an edge encoding representing a type of bond between a pair of the normal nodes in each pre-transformation molecular graph, and a spatial encoding representing a shortest path distance along the edges between the pair of the normal nodes in each pre-transformation molecular graph. The processor may be further configured to input the training data set to a transformer-based graph neural network to thereby train the transformer-based graph neural network to perform an inference at inference time. To perform the inference at inference time, the processor may be further configured to receive inference-time input of an inference-time pre-transformation molecular graph at the transformer-based graph neural network, and output the inference-time post-transformation energy parameter value based on the inference-time pre-transformation molecular graph.

According to this aspect, the encoded structural information may include a centrality encoding embedding for at least one of the normal nodes of each pre-transformation molecular graph.

According to this aspect, the centrality encoding may be a degree of the at least one normal node of each pre-transformation molecular graph.

According to this aspect, the centrality encoding may assign the at least one normal node two real-valued embedding vectors according to an indegree and an outdegree of the respective normal node.

According to this aspect, the shortest path distance represented by the spatial encoding may be a weighted shortest path distance.

According to this aspect, each pre-transformation molecular graph further may include one virtual node fully connected by virtual edges to all normal nodes of the respective pre-transformation molecular graph.

According to this aspect, the encoded structural information may be represented as a learnable scalar bias term in a self-attention layer of an encoder of the transformer of the transformer-based graph neural network.

According to this aspect, the energy transformation may be due to molecular relaxation of the molecular system.

According to another aspect of the present disclosure, a computerized method is provided. The computerized method may include, during a training phase, providing a training data set including a plurality of training data pairs, each of the training data pairs including a pre-transformation molecular graph and post-transformation energy parameter value representing an energy change in a molecular system following an energy transformation, in which the pre-transformation graph includes a plurality of normal nodes connected by edges, and each normal node represents an atom in the molecular system. The computerized method may further include encoding structural information in each pre-transformation molecular graph as learnable embeddings, in which the structural information describes the relative positions of the atoms represented by the normal nodes. The structural information may include an edge encoding representing a type of bond between a pair of the normal nodes in each pre-transformation molecular graph, and a spatial encoding representing a shortest path distance along the edges between the pair of the normal nodes in each pre-transformation molecular graph. The computerized method may further include inputting the training data set to a transformer-based graph neural network to thereby train the transformer-based graph neural network to perform an inference at inference time. To perform the inference at inference-time, the computerized method may further include receiving inference-time input of an inference-time pre-transformation molecular graph at the transformer-based graph neural network, and outputting the inference-time post-transformation energy parameter value based on the inference-time pre-transformation molecular graph.

According to this aspect, the encoded structural information may include a centrality encoding embedded for at least one normal node of each pre-transformation molecular graph.

According to this aspect, the centrality encoding may be a degree of the at least one normal node of each pre-transformation molecular graph.

According to this aspect, the centrality encoding may assign the at least one normal node two real-valued embedding vectors according to an indegree and an outdegree of the respective normal node.

According to this aspect, the shortest path distance represented by the spatial encoding may be a weighted shortest path distance.

According to this aspect, each molecular graph further may include one virtual node fully connected by virtual edges to all normal nodes of the respective pre-transformation molecular graph.

According to this aspect, the encoded structural information may be represented as a learnable scalar bias term in a self-attention layer of an encoder of the transformer of the transformer-based graph neural network.

According to this aspect, the energy transformation may be due to molecular relaxation of the molecular system.

According to another aspect of the present disclosure, a computing system is provided. The system may include a processor configured to, during a training phase, provide a training data set including a plurality of training data pairs, each of the training data pairs including a pre-transformation graph and post-transformation parameter value representing a change in a system modeled by the pre-transformation graph following a transformation, in which the pre-transformation graph may include a plurality of normal nodes connected by edges, and each normal node may represent a location in the system. The processor may be further configured to encode structural information in each pre-transformation graph as learnable embeddings, in which the structural information describes the relative positions of the locations represented by the normal nodes. The structural information may include an edge encoding representing a type of connection between a pair of the normal nodes in each pre-transformation graph, and a spatial encoding representing a shortest path distance along the edges between the pair of the normal nodes in each pre-transformation graph. The processor may be further configured to input the training data set to a transformer-based graph neural network to thereby train the transformer-based graph neural network to perform an inference at inference time.

According to this aspect, the pre-transformation graph may be a social graph that models a social network of friends, a map that models a network of locations connected by roads or railways, or a knowledge graph that models knowledge sources connected by references.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A computing system, comprising:
a processor configured to:
during a training phase,
provide a training data set including a plurality of training data pairs, each of the training data pairs including a pre-transformation molecular graph and post-transformation energy parameter value representing an energy change in a molecular system following an energy transformation, wherein the pre-transformation molecular graph includes a plurality of normal nodes connected by edges, each normal node representing an atom in the molecular system;
encode structural information in each pre-transformation molecular graph as learnable embeddings, the structural information describing the relative positions of the atoms represented by the normal nodes, the structural information including:
an edge encoding representing a type of bond between a pair of the normal nodes in each pre-transformation molecular graph; and
a spatial encoding representing a shortest path distance along the edges between the pair of the normal nodes in each pre-transformation molecular graph; and
input the training data set to a transformer-based graph neural network to thereby train the transformer-based graph neural network to perform an inference at inference time.

2. The computing system of claim 1, wherein to perform the inference at inference time, the processor is further configured to,
receive inference-time input of an inference-time pre-transformation molecular graph at the transformer-based graph neural network, and
output the inference-time post-transformation energy parameter value based on the inference-time pre-transformation molecular graph.

3. The computing system of claim 1, wherein the encoded structural information includes a centrality encoding embedding for at least one of the normal nodes of each pre-transformation molecular graph.

4. The computing system of claim 3, wherein the centrality encoding is a degree of the at least one normal node of each pre-transformation molecular graph.

5. The computing system of claim 4, wherein the centrality encoding assigns the at least one normal node two real-valued embedding vectors according to an indegree and an outdegree of the respective normal node.

6. The computing system of claim 1, wherein the shortest path distance represented by the spatial encoding is a weighted shortest path distance.

7. The computing system of claim 1, wherein each pre-transformation molecular graph further includes one virtual node fully connected by virtual edges to all normal nodes of the respective pre-transformation molecular graph.

8. The computing system of claim 1, wherein the encoded structural information is represented as a learnable scalar bias term in a self-attention layer of an encoder of the transformer of the transformer-based graph neural network.

9. The computing system of claim 1, wherein
the energy transformation is due to molecular relaxation of the molecular system.

10. A computerized method, comprising:
during a training phase,
providing a training data set including a plurality of training data pairs, each of the training data pairs including a pre-transformation molecular graph and post-transformation energy parameter value representing an energy change in a molecular system following an energy transformation, wherein the pre-transformation graph includes a plurality of normal nodes connected by edges, each normal node representing an atom in the molecular system;
encoding structural information in each pre-transformation molecular graph as learnable embeddings, the structural information describing the relative positions of the atoms represented by the normal nodes, the structural information including:
an edge encoding representing a type of bond between a pair of the normal nodes in each pre-transformation molecular graph; and
a spatial encoding representing a shortest path distance along the edges between the pair of the normal nodes in each pre-transformation molecular graph; and
inputting the training data set to a transformer-based graph neural network to thereby train the transformer-based graph neural network to perform an inference at inference time.

11. The computerized method of claim 10, further comprising:
to perform the inference at inference-time,
receiving inference-time input of an inference-time pre-transformation molecular graph at the transformer-based graph neural network; and
outputting the inference-time post-transformation energy parameter value based on the inference-time pre-transformation molecular graph.

12. The computerized method of claim 10, wherein the encoded structural information includes a centrality encoding embedded for at least one of the normal nodes of each pre-transformation molecular graph.

13. The computerized method of claim 12, wherein the centrality encoding is a degree of the at least one normal node of each pre-transformation molecular graph.

14. The computerized method of claim 13, wherein the centrality encoding assigns the at least one normal node two real-valued embedding vectors according to an indegree and an outdegree of the respective normal node.

15. The computerized method of claim 14, wherein the shortest path distance represented by the spatial encoding is a weighted shortest path distance.

16. The computerized method of claim 10, wherein each molecular graph further includes one virtual node fully connected by virtual edges to all normal nodes of the respective pre-transformation molecular graph.

17. The computerized method of claim 10, wherein the encoded structural information is represented as a learnable scalar bias term in a self-attention layer of an encoder of the transformer of the transformer-based graph neural network.

18. The computerized method of claim 10, wherein
the energy transformation is due to molecular relaxation of the molecular system.

19. A computing system, comprising:
a processor configured to:
during a training phase,
provide a training data set including a plurality of training data pairs, each of the training data pairs including a pre-transformation graph and post-transformation parameter value representing a change in a system modeled by the pre-transformation graph following a transformation, wherein the pre-transformation graph includes a plurality of normal nodes connected by edges, each normal node representing a location in the system;
encode structural information in each pre-transformation graph as learnable embeddings, the structural information describing the relative positions of the locations represented by the normal nodes, the structural information including:
an edge encoding representing a type of connection between a pair of the normal nodes in each pre-transformation graph; and
a spatial encoding representing a shortest path distance along the edges between the pair of the normal nodes in each pre-transformation graph; and
input the training data set to a transformer-based graph neural network to thereby train the transformer-based graph neural network to perform an inference at inference time.

20. The computing system of claim 19, wherein the pre-transformation graph is a social graph that models a social network of friends, a map that models a network of locations connected by roads or railways, or a knowledge graph that models knowledge sources connected by references.
